(19) United States
(12) Patent Application Publication
(10) Pub. No.: US 2010/0190854 A1
(43) Pub. Date: Jul. 29, 2010

(54) Composition and method to treat mastitis

(76) Inventor: Anthony J. Harris, Sun City, AZ (US)

Correspondence Address: TOD R NISSLE, PO BOX 55630, PHOENIX, AZ, 85078, US

(21) Appl. No.: 12/587,562

(22) Filed: Oct. 8, 2009

Related U.S. Application Data
(60) Provisional application No. 61/195,769, filed on Oct. 10, 2008.

(51) Int. Cl. A61K 31/23; A61P 29/00

(52) U.S. Cl. 514/533

(57) ABSTRACT

A method is provided to produce a treatment composition which is sprayed externally on to the udder of a cow to treat mastitis.

This application Claims priority based on provisional patent application 61/195769, filed Oct. 10, 2008.

This invention relates to the treatment of mastitis.

Bovine mastitis is an inflammation of the mammary gland or udder of a cow. Bovine mastitis is caused by a variety of pathogens including, for example, Pseudocowpox and Herpes mammilitis. When a cow has mastitis, sores or lesions appear on teat skin tissue. These lesions often can be quite large and can extend down to the teat end. Once lesions appear there is no available remedy that quickly heals up the skin. Conventional treatment consists of applying an iodine/glycerine solution that facilitates healing and prevents the occurrence of secondary bacterial infections. During the several week period required for recovery, the mastitis may progress through a herd over a period of weeks or months.

Accordingly, it would be highly desirable to provide an improved method and composition for treating bovine mastitis.

Therefore, it is a principal object of the instant invention to provide an improved method and composition for treating mastitis.

I have discovered a spray composition for treating bovine mastitis. The composition is produced by first admixing the below components to produce a preliminary composition:

| Component | Weight Percent |
|---|---|
| Caprylic Acid and Caprylic Acid Triglyceride | 50% to 70% (by weight) |
| Coco Caprylate/Caprate | 20% to 35% |
| C18-36 Acid Triglyceride | 1.0% to 4.0% |
| C18-36 Acid Glycol Ester | 0.5% to 2.0% |
| Tribehenin | 0.001% to 0.9% |
| Beeswax | 0.001% to 0.9% |
| Eucalyptus Oil | 0.001% to 0.9% |
| D-Alpha Bisobolol | 0.001% to 0.9% |
| D-Alpha Tocopheryl | 0.001% to 0.9% |
| Tea Tree Oil | 0.001% to 0.9% |
| Peppermint Oil | 0.001% to 0.9% |
| Aloe Vera Gel | 0.001% to 0.9% |
| Frescolate ML | 0.001% to 0.9% |
| Gingko Biloba | 0.001% to 0.9% |

The foregoing ingredients are admixed utilizing conventional mixing procedures to produce the preliminary composition. Distilled water, alcohol, or another liquid is admixed with the preliminary composition in a quantity sufficient to produce a secondary composition having a viscosity that enables the secondary composition to be sprayed onto the udder of a cow.

Medium chain triglycerides (MCTs) are medium-chain (6 to 12 carbons) fatty acid esters of glycerol. Coconut oil contains about 66% MCTs. MCTs include fatty acids called medium chain fatty acids. Medium chain fatty acides (and the crresponding number of carbons) found in MCTs are: caproic acid (C6), caprylic acid (C8), caprric acid (C10) and lauric acid (C12). A MCT is comprised of glycerol and three of the listed fatty acids.

Octoanoic acid is an eight-carbon saturated fatty acid also known as caprylic acid. It occurs naturally in breast milk and coconuts and is an oily liquid that is only slightly soluble in water. Caprylic acid is used in the treatment of some bacterial infections. It has a fairly short chain length and, consequently, can penetrate fatty cell wall membranes to attack lipid-coated bacteria like Staphylococcus aureus and species of Streptococcus. Caprylic acid is used as a food contact surface sanitizer in commercial food handling businesses such as beverage processing plants, breweries, and wineries, and is used on dairy equipment and food processing equipment. A variety of establishments have used it as a disinfectant, including hotels, retail and wholesale stores, livestock lots, recreational facilities, office buildings, restaurants, colleges, health care facilities, and animal care/veterinary facilities. Further, greenhouses, garden centers, and nurseries utilize caprylic acid as an algaecide, bactericide, and fungicide.

Tribehenin is a glyceryl tribehenate or glyceryl triester. It includes glycerin and behenic acid. Glycerin is a humectant and a smoothing lubricant. Behenic acid is a conditioner and is a fatty acid that is derived from plants. Tribehenin is used in many skin care products as a conditioning agent. Such products include creams, lotions, skin cleaning products, makeup formulas, and sunscreen products. Tribehenin is found in animal and vegetable fats and oils including tallow, coconut oils, and palm nut oils.

The following example is provided by way of illustration, and not limitation, of the invention.

### EXAMPLE I

The following components are provided in the indicated weight percentages.

| Component | Weight Percent |
|---|---|
| Caprylic Acid and Caprylic Acid Triglyceride | 65 |
| Coco Caprylate/Caprate | 30 |
| C18-36 Acid Triglyceride | 2.5 |
| C18-36 Acid Glycol Ester | 1.0 |
| Tribehenin | 0.2 |
| Beeswax | 0.2 |
| Eucalyptus Oil | 0.2 |
| D-Alpha Bisobolol | 0.2 |
| D-Alpha Tocopheryl | 0.2 |
| Tea Tree Oil | 0.1 |
| Peppermint Oil | 0.1 |
| Aloe Vera Gel | 0.1 |
| Frescolate ML | 0.1 |
| Gingko Biloba | 0.1 |

These foregoing components are mixed together to form a preliminary composition. The preliminary composition is mixed together with a secondary composition of 50% water and 50% ethanol to produce a final treatment composition having a viscosity suitable for spraying. As can be appreciated by those of skill in the art, the proportion of water to alcohol can be varied as desired, and any desired alcohol or other composition can be utilized.

A first cow is provided which has mastitis on its udder, including sores or lesions that appear on the teat skin tissue. The final treatment composition is applied twice daily, i.e., each time the cow is fed: once in the morning and once in the afternoon. At each application an amount of the final composition is sprayed that is sufficient to wet substantially the entire outer surface of the udder. After ten days the lesions are gone and the teat skin tissue has healed.

### EXAMPLE II

Example I is repeated, except a second cow is provided which has mastitis on its udder, including sores or lesions that appear on the teat skin tissue. The final composition is applied twice daily, i.e., each time the cow is fed: once in the morning and once in the afternoon. At each application an amount of the final composition is sprayed that is sufficient to wet substantially the entire outer surface of the udder. After eight days the lesions are gone and the teat skin tissue has healed.

### EXAMPLE III

Example I is repeated, except a third cow is provided which has mastitis on its udder, including sores or lesions that appear on the teat skin tissue. Similar results are obtained.

### EXAMPLE IV

Example I is repeated, except a fourth cow is provided which has mastitis on its udder, including sores or lesions that appear on the teat skin tissue, and the treatment composition is sprayed on the cow's udder only once a day. After fourteen days the lesions are gone and the teat skin tissue has healed.

The methodology of the invention can be utilized on the mammary glands of human beings or animals other than cows.

Having described the presently preferred embodiments and best mode of the invention in such terms as to enable those of skill in the art to understand and practice the invention, I Claim:

1. A method to treat mastitis on the udder of a cow, including
(a) providing a preliminary composition with the following components in the indidcated weight percents:

| | |
|---|---|
| Caprylic Acid and Caprylic Acid Triglyceride | 50% to 70% |
| Coco Caprylate/Caprate | 20% to 35% |
| C18-36 Acid Triglyceride | 1.0% to 4.0% |
| C18-36 Acid Glycol Ester | 0.5% to 2.0% |
| Tribehenin | 0.001% to 0.9% |
| Beeswax | 0.001% to 0.9% |
| Eucalyptus Oil | 0.001% to 0.9% |
| D-Alpha Bisobolol | 0.001% to 0.9% |
| D-Alpha Tocopheryl | 0.001% to 0.9% |
| Tea Tree Oil | 0.001% to 0.9% |
| Peppermint Oil | 0.001% to 0.9% |
| Aloe Vera Gel | 0.001% to 0.9% |
| Frescolate ML | 0.001% to 0.9% |
| Gingko Biloba | 0.001% to 0.9% |

(b) said primary composition components are admixed with water and alcohol to produce a sprayable treatment composition; and,
(c) said sprayable composition is sprayed on the udder of the cow at least once a day for a selected period of time for eight to ten days.
